(19) United States
(12) Patent Application Publication
(10) Pub. No.: US 2026/0115397 A1
(43) Pub. Date: Apr. 30, 2026

(54) RESPIRATORY TREATMENT SUPPORT SYSTEM, SERVER, AND DISPLAY DEVICE

(71) Applicant: FUKUDA DENSHI CO., LTD., Tokyo (JP)

(72) Inventors: Eriko Yoshioka, Tokyo (JP); Eiji Takahashi, Tokyo (JP)

(21) Appl. No.: 19/365,259

(22) Filed: Oct. 22, 2025

(30) Foreign Application Priority Data
Oct. 31, 2024 (JP) 2024-192057

(51) Int. Cl. A61M 16/00; G16H 40/67

(52) U.S. Cl. CPC A61M 16/024; G16H 40/67; A61M 2016/0027; A61M 2205/15; A61M 2205/3584; A61M 2205/502

(57) ABSTRACT

A respiratory treatment support system according to the present disclosure includes: a respiratory treatment device; a server communicably connected to the respiratory treatment device; and an information terminal communicably connected to the server, in which the server receives operational information on the respiratory treatment device from the respiratory treatment device, stores the operational information and calculates a statistical value and a histogram of the operational information, and is capable of transmitting the statistical value and the histogram to the information terminal.

### CROSS REFERENCE TO RELATED APPLICATIONS

This application is entitled to and claims the benefit of Japanese Patent Application No. 2024-192057, filed on October 31, 2024, the disclosure of which including the specification, drawings and abstract is incorporated herein by reference in its entirety.

### TECHNICAL FIELD

The present disclosure relates to, for example, a respiratory treatment support system, a server, and a display device that support treatment using a respiratory treatment device such as a continuous positive airway pressure (CPAP) device.

### BACKGROUND ART

Home treatment using a respiratory treatment device such as a CPAP device or an oxygen concentrator has been widely performed.

In addition, a home medical care support system has been proposed in which this respiratory treatment device and a medical institution such as a hospital are communicably connected to each other via a server (for example, see Patent Literature (hereinafter, referred to as PTL) 1). This type of home medical care support system stores operational information of the home medical equipment in the server. In the home medical care support system, a healthcare professional such as a physician can access the server from an information terminal such as a personal computer, and can check the operational information of the respiratory treatment device at any time. In addition, in the home medical care support system, the healthcare professional can also transmit setting information or the like of the respiratory treatment device to the respiratory treatment device via the server.

### Citation List

### Patent Literature

### PTL 1

Japanese Patent Application Laid-Open No. 2023-061121

Incidentally, in the conventional service of a respiratory treatment support system in which a CPAP device is connected, the operational information of the CPAP device can be checked on the information terminal operated by a physician or the like. This operational information is, for example, a usage duration of the CPAP device, a value of pressure supplied to a patient, a value of leak, and/or the like.

Incidentally, in the conventional respiratory treatment support system, the above-described operational information can be viewed on the information terminal used by a physician or the like in the form of statistical values such as mean values or median values.

However, the above statistical values provided to the physician or the like are not necessarily appropriate statistical values for the physician to determine a treatment plan or the like. It is difficult for the physician to assess the reliability of the provided statistical values, which may consequently make it challenging to determine an appropriate treatment plan promptly.

The present disclosure has been made in consideration of the above points, and provides a respiratory treatment support system, a server, and a display device that enable a healthcare professional such as a physician to more accurately recognize an operation of a respiratory treatment device.

### SUMMARY

One aspect of a respiratory treatment support system of a present disclosure includes: a respiratory treatment device; a server communicably connected to the respiratory treatment device; and an information terminal communicably connected to the server. In the respiratory treatment support system, the server receives operational information on the respiratory treatment device from the respiratory treatment device, the server stores the operational information and calculates a statistical value and a histogram of the operational information, and the server is capable of transmitting the statistical value and the histogram to the information terminal.

One aspect of a server of the present disclosure is used in a respiratory treatment support system including: a respiratory treatment device; the server communicably connected to the respiratory treatment device; and an information terminal communicably connected to the server. The server receives operational information on the respiratory treatment device from the respiratory treatment device, stores the operational information, and calculate a statistical value and a histogram of the operational information, and can transmit the statistical value and the histogram to the information terminal.

One aspect of a display device of the present disclosure is used in a respiratory treatment support system including; a respiratory treatment device; a server communicably connected to the respiratory treatment device; and an information terminal communicably connected to the server. The display device receives a statistical value and a histogram of operational information on the respiratory treatment device from the server, and can display the statistical value and the histogram.

### BRIEF DESCRIPTION OF THE DRAWINGS

FIG. 1 is a schematic diagram illustrating an overall respiratory treatment support system according to an embodiment;

FIG. 2 is a diagram illustrating a display example of a summary of operational information displayed on a PC (information terminal);

FIG. 3 is a diagram illustrating an example of setting image displayed on the PC (information terminal);

FIG. 4 is a diagram illustrating a display example in a case where a histogram is approximately normally distributed;

FIG. 5 is a diagram illustrating a display example in a case where the histogram is not normally distributed; and

FIG. 6 is a block diagram illustrating a configuration example of a server for realizing the provision of the operational information of the embodiment.

### DESCRIPTION OF EMBODIMENTS

Hereinafter, an embodiment of the present disclosure will be described in detail with reference to the drawings.

### 1 Overall Configuration of System

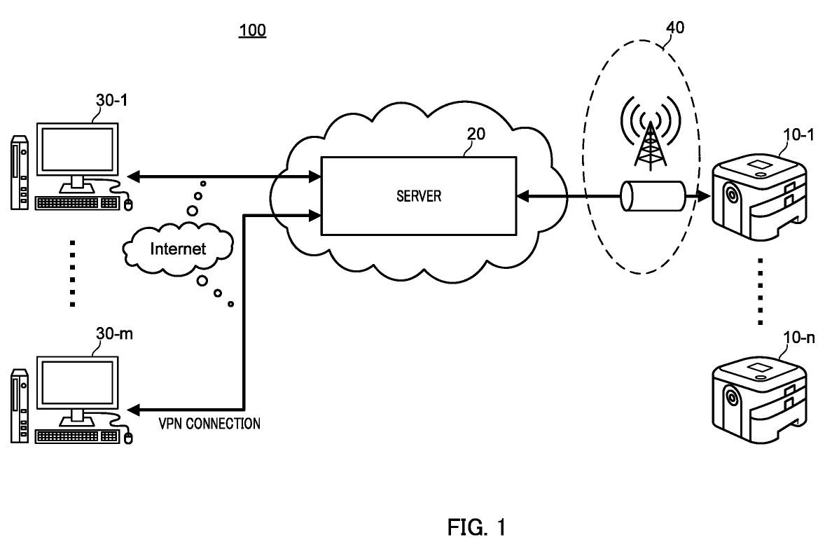

FIG. 1 is a schematic diagram illustrating an overall respiratory treatment support system according to the present embodiment.

Respiratory treatment support system 100 includes a plurality of continuous positive airway pressure (CPAP) devices 10-1 to 10-n (n is an integer of 2 or more), server 20, and personal computers (hereinafter, abbreviated as “PC”) 30-1 to 30-m (m is an integer of 1 or more) as information terminals and display devices. Note that, in the following description, CPAP devices 10-1 to 10-n are sometimes abbreviated as CPAP devices 10. In addition, PCs 30-1 to 30-m are sometimes abbreviated as PCs 30.

Each of CPAP devices 10 is a device that prevents apnea in a patient during sleep by continuously delivering air to the airway of the patient with obstructive sleep apnea to widen the airway. Each of CPAP devices 10 is provided, for example, in the home of each patient.

Server 20 receives and stores information transmitted from the plurality of CPAP devices 10. In the example of the present embodiment, server 20 is constructed on the cloud, and functions as a cloud server that provides information to the plurality of PCs 30 via a network such as the Internet (registered trademark).

Server 20 is realized by, for example, a computer including hardware resources such as a processor and a memory, and is configured such that the processor executes a program for realizing each function according to the present disclosure.

PCs 30 are provided in a medical institution such as a hospital, a management company that manages CPAP devices 10, or the like. PCs 30 can access server 20 via the Internet and read out treatment data and the like stored in server 20. In addition, at least some of PCs 30 may be capable of transmitting setting information or the like to CPAP devices 10 via server 20.

Here, CPAP devices 10 and server 20 are communicably connected to each other via closed communication network 40. Closed communication network 40 may be wireless or wired. Using closed communication network 40 avoid leakage of communication data between CPAP devices 10 and server 20. Using closed communication network 40 can also prevent an unintended terminal from accessing server 20 and tampering with treatment data. Consequently, the reliability of the treatment data stored in server 20 can be improved.

In the example of FIG. 1, an example is shown in which CPAP devices 10 are connected to closed communication network 40. However, in addition to CPAP device 10, for example, another respiratory treatment device such as an oxygen concentrator or a ventilator may be communicably connected to server 20. Furthermore, another medical equipment such as a physiological monitor may be communicably connected to server 20.

### 2 Provision of Operational Information

Next, the operational information of CPAP device 10 provided from server 20 to PC 30 as an information terminal will be described.

Here, examples of information (data) transmitted from CPAP device 10 to server 20 include pressure data, equipment information, a respiratory event, summary data, environment information, setting (prescription) information, reminder information, a data time, an event log, an error log, and network time protocol (NTP) information.

The pressure data is data on the pressure of air delivered from CPAP device 10 to the airway of the patient. CPAP device 10 treats the apnea of the patient with this pressure, and thus the pressure data is treatment data for CPAP device 10.

The equipment information is operating information of CPAP device 10, the respiratory event is event information such as the apnea of the patient, the summary data is single treatment summary information, the environment information is temperature and humidity inside the equipment or the like, the setting (prescription) information is setting information of the equipment (CPAP device 10), the reminder information (next visit date) is a latest next visit date, the data time is a latest time of various transmission data, the event log is a log indicating an operation content of the equipment, the error log is error occurrence information, and the NTP information is a current time.

Among the data transmitted from CPAP device 10 to server 20, the pressure data, which is the treatment data, is mainly transmitted during the treatment, and the other data are mainly transmitted when an AC power is turned on, at the start of the treatment, when the treatment is not performed, or at the end of the treatment. However, the data transmitted during the treatment is not limited to the pressure data, and the environment information, the event log, the error log, and the like may also be transmitted during the treatment.

Server 20 calculates statistical values and the like of the operational information of CPAP device 10 based on the above-described data received from CPAP device 10. Here, the operational information is, for example, a usage duration of CPAP device 10, a value of pressure supplied to the patient, a value of leak, and/or the like, and the statistical values of the operational information are mean values and median values of these values. Server 20 transmits the calculated statistical values of the operational information to PC 30 in response to a request from PC 30.

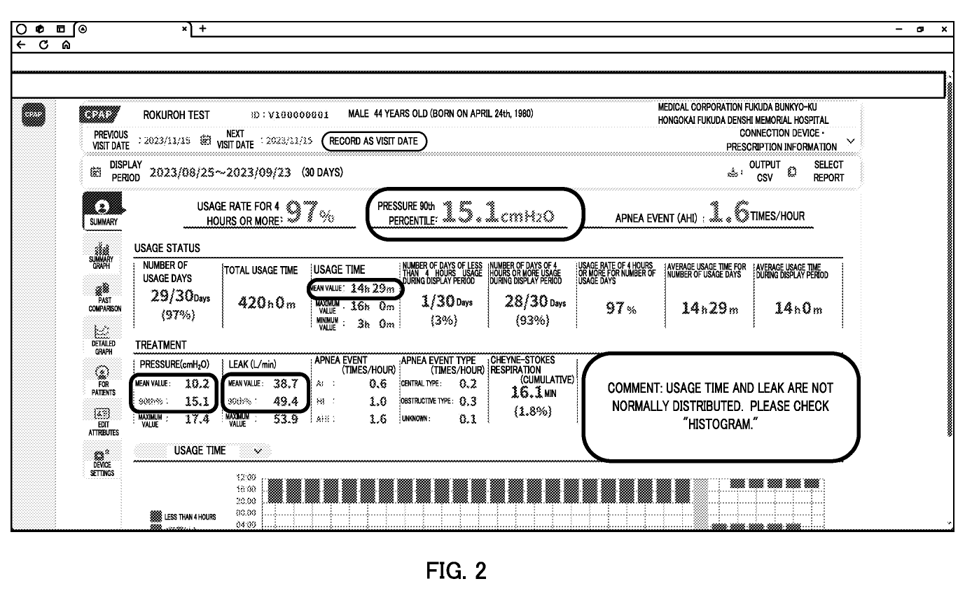

FIG. 2 illustrates a display example of a summary of the operational information displayed on PC 30. Note that thick square lines surrounding “pressure 90th percentile: 15.1 cmH₂O,” “mean value: 14 h 29 m,” “mean value: 10.2, 90th percentile: 15.1,” and “mean value: 38.7, 90th percentile: 49.4” are added for convenience of description and are not actually displayed.

The summary of the operational information of CPAP device 10 is provided from server 20 to PC 30 as, for example, statistical values of the operation for 10 to 120 days. In the example of FIG. 2, the statistical values of the operation for 30 days are provided to PC 30 as a summary for 30 days and are displayed on PC 30. Note that the operation period (30 days in the example of FIG. 2) for calculating the statistical values may be set on a day-by-day basis by, for example, displaying a calendar.

In addition, in the example of FIG. 2, the mean value and the 90th percentile (percentile value) are displayed as the statistical values. Specifically, the summary display in FIG. 2 includes a 90th percentile pressure value (15.1 cmH₂O in FIG. 2), a mean value of the usage duration (14 h 29 m in FIG. 2), a mean value of the pressure (10.2 (cmH₂O) in FIG. 2), a mean value of the leak (38.7 (L/min) in FIG. 2), a 90th percentile leak value (49.4 (L/min) in FIG. 2), and the like.

In respiratory treatment support system 100 of the present embodiment, the mean value and the median value can be switched and displayed as the statistical value. In addition, the percentile values can be switched and displayed between the 90th percentile and the 95th percentile.

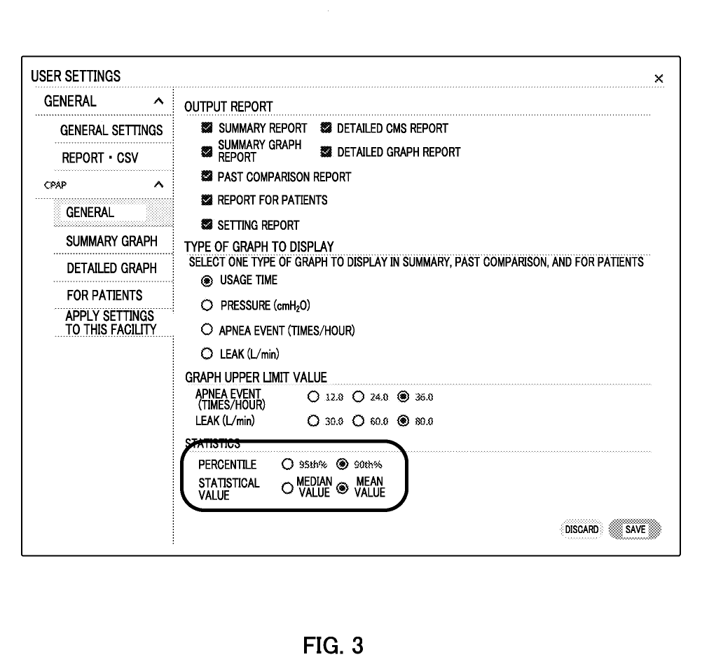

This will be described in detail. FIG. 3 illustrates an example of a settings image displayed on PC 30 used by a user such as a physician. As can be seen from FIG. 3, the setting image displays the 90th percentile and the 95th percentile as the percentile values, and displays the mean value and the median value as the statistical values. Note that the percentile value is also one of the statistical values. As the statistical value, a mode value or the like may also be displayed. The user selects, for example, the 90th percentile or the 95th percentile as the percentile value by operating a mouse, and further selects the mean value or the median value. The selection result is transmitted to server 20, and the statistical values corresponding to the selection result are transmitted from server 20 to PC 30.

That is, the display example in FIG. 2 is an example of a display in a case where the 90th percentile value and the mean value are selected as the statistical values by the user. On the other hand, for example, in a case where the 95th percentile value and the median value are selected as the statistical values by the user, numerical values corresponding to the selection result are displayed on PC 30.

Furthermore, in the present embodiment, server 20 calculates a histogram of the operational information and transmits the histogram to PC 30.

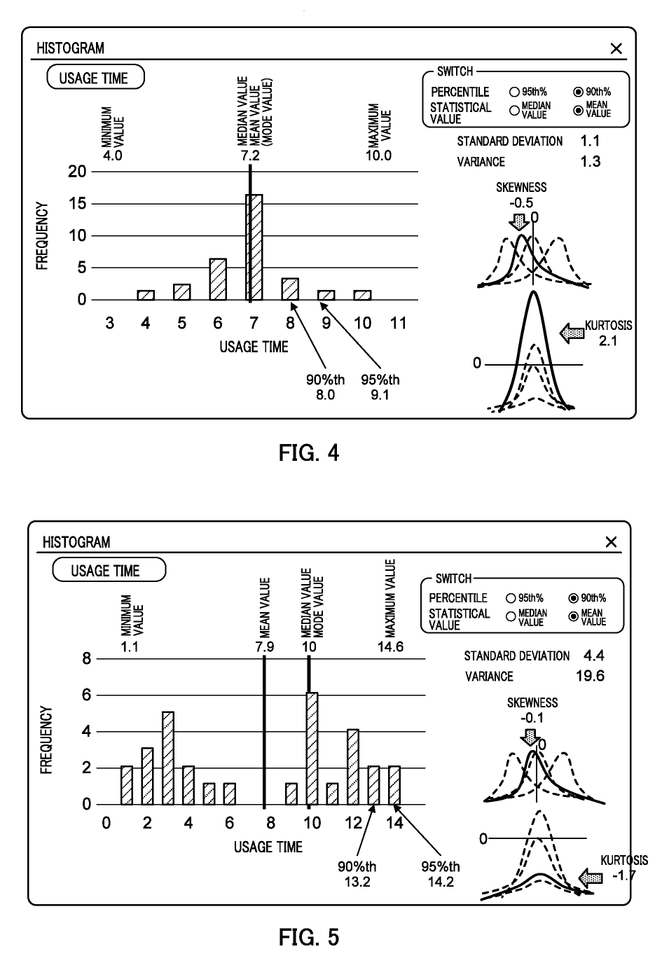

FIGS. 4 and 5 each illustrate a display example of the histogram on PC 30. In the examples of the figures, a usage time histogram is displayed. In addition, in the examples of the figures, graphs of skewness and kurtosis are displayed at the lower right of the screen. In these graphs of skewness and kurtosis, the solid line represents a graph corresponding to the histogram, and the dotted line represents a graph for comparison.

By viewing the histogram, the user, such as a physician, can check the positions of the mean value and the median value relative to the overall distribution, as well as the positions of the 90th percentile and the 95th percentile relative to the overall distribution. As a result, the user easily selects which of the mean value or the median value should be displayed or whether both the mean value and the median value should be displayed, from the relationship between the histogram and the mean value and the median value. In addition, the user can recognize which of the mean value or the median value should be considered or emphasized.

Similarly, the user easily selects which of the 90th percentile or the 95th percentile should be displayed or whether both the 90th percentile and the 95th percentile should be displayed, from the relationship between the histogram and the 90th percentile and the 95th percentile. In addition, the user can recognize which of the 90th percentile or the 95th percentile should be considered or emphasized.

FIG. 4 is an example in which the histogram is approximately normally distributed, and FIG. 5 is an example in which the histogram is not normally distributed. In a case where the histogram is approximately normally distributed as in FIG. 4, the mean value and the median value have substantially the same value, so that the user can recognize that the values are substantially the same regardless of which of the mean value or the median value is selected. For example, in the example of FIG. 4, because the values of both the mean value and the median value are “7.2,” and the horizontal axis positions (lines extending in the vertical direction) indicated by the values match and overlap with each other, the user can recognize that the values of both the mean value and the median value are substantially the same.

In contrast, in a case where the histogram is not normally distributed as in FIG. 5, the mean value and the median value have significantly different values, so that the user can recognize that the values are significantly different depending on which of the mean value or the median value is selected. For example, in the example of FIG. 5, because the mean value is “7.9,” the median value is “10,” and the horizontal axis positions (lines extending in the vertical direction) indicated by the values are separated from each other, the user can recognize that the mean value and the median value are separated from each other.

Furthermore, in the display examples of FIGS. 4 and 5, a standard deviation, a variance, the skewness, and the kurtosis of the histogram are displayed. This allows the user to more easily recognize the shape of the histogram and can use the shape as an indicator for selecting which of the mean value and/or the median value to be selected.

In addition, for example, in the example of FIG. 4, the user can recognize that the 90th percentile value is “8.0” and the 95th percentile value is “9.1” on a single screen, and for example, in the example of FIG. 5, the user can recognize that the 90th percentile value is “13.2” and the 95th percentile value is “14.2” on the single screen. This allows the user to easily recognize the statistical values of the 90th percentile and the 95th percentile on the histogram and can use the statistical values as indicators for selecting which of the 90th percentile or the 95th percentile to be selected.

Furthermore, in the display examples of FIGS. 4 and 5, in addition to the histogram, the mean value, the median value, and the percentile values, a settings image for switching between the 90th percentile and the 95th percentile and for switching between the mean value and the median value is displayed. In the examples of FIGS. 4 and 5, the settings image is displayed in a “switch” area at a position in the upper right of the screen. The user can select and switch the percentile value by, for example, moving a mouse cursor to any of the displayed 90th percentile and 95th percentile and performing a click operation. Similarly, the user can select and switch to the median value or the mean value by, for example, moving the mouse cursor to any of the displayed median value and mean value and performing a click operation.

Accordingly, the user can easily switch between the mean value and the median value and switch the percentile value while viewing the relationship between the histogram, the mean value, the median value, and the percentile values. Note that, among the median value, the mean value, the 95th percentile, and the 90th percentile displayed in the “switch” area on the screen in FIGS. 4 and 5, the selected item may be emphasized.

As described above, in the present embodiment, the statistical values can be selected and switched not only on the settings screen illustrated in FIG. 3 but also on the screen of the histogram illustrated in FIGS. 4 and 5, and the selected and switched statistical values can be reflected in the summary in FIG. 2.

Noe that, in the examples of FIGS. 4 and 5, the case has been described where the histogram, the mean value, the median value, and the percentile value of the usage duration of CPAP device 10 are displayed among the operational information. However, the histogram, the mean value, the median value, and the percentile value of the pressure or the leak of CPAP device 10 may be displayed among the operational information.

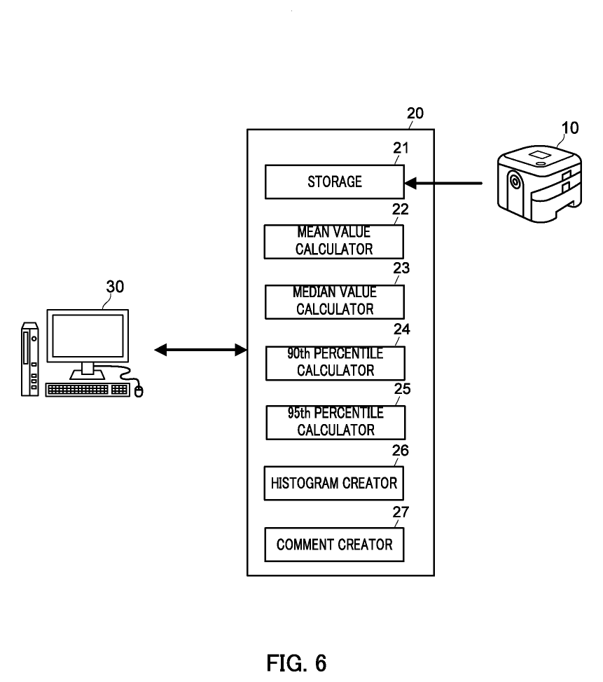

FIG. 6 is a block diagram illustrating a configuration example of server 20 for realizing the provision of the above-described operational information. Server 20 includes storage 21, mean value calculator 22, median value calculator 23, 90th percentile calculator 24, 95th percentile calculator 25, histogram creator 26, and comment creator 27. The functions of server 20 are realized by, for example, hardware such as a processor and a memory.

The operational information (the usage duration, the pressure value, the leak value, and the like) transmitted from CPAP device 10 is stored in storage 21. Mean value calculator 22 calculates an mean value of each piece of the operational information, median value calculator 23 calculates a median value of each piece of the operational information, 90th percentile calculator 24 calculates a 90th percentile value of each piece of the operational information, 95th percentile calculator 25 calculates a 95th percentile value of each piece of the operational information, and histogram creator 26 creates a histogram of each piece of the operational information. The obtained mean value, the median value, the 90th percentile value, the 95th percentile value, and the histogram of the operational information are transmitted to PC 30 as necessary.

Mean value calculator 22, median value calculator 23, 90th percentile calculator 24, and 95th percentile calculator 25 read out the operational information stored in storage 21 and perform the calculation. The number of source data items used in the various calculations performed by mean value calculator 22, median value calculator 23, 90th percentile calculator 24, and 95th percentile calculator 25 varies depending on the number of days of the display period. Specifically, the data and the number of data items to be read by mean value calculator 22, median value calculator 23, 90th percentile calculator 24, and 95th percentile calculator 25 may vary based on at least any one of a setting value of “Display period: ... (for 30 days)” shown in the upper left of the summary in FIG. 2, “Number of usage days ...” shown in the left center or “Number of days of ... usage during display period” shown in the center in FIG. 2.”

Comment creator 27 creates a comment based on the histogram created by histogram creator 26. The created comment is transmitted to PC 30. This will be described in detail.

Comment creator 27 creates a comment based on a frequency distribution of the histogram created by histogram creator 26, and transmits information on the comment to PC 30. Examples of the comment include “The usage time and the leak are not normally distributed. Please check the ‘histogram’.” As illustrated in FIG. 2, this comment is displayed on PC 30.

When the user such as a physician who has seen the comment clicks on the text “histogram” in FIG. 2, the histogram as illustrated in FIGS. 4 and 5 is transmitted from server 20 to PC 30 to be displayed. In short, the comment transmitted from server 20 to PC 30 in this case is information that prompts the user of PC 30 to select and display the histogram.

That is, the user of PC 30 has difficulty to determine whether the mean value or the median value is appropriate. Then, when the histogram is not normally distributed, server 20 displays a comment that prompts the user of PC 30 to check the histogram. The comment is not limited to the one prompting the user to check the histogram, and may be, for example, a comment that prompts the user to check both the mean value and the median value.

Note that the comment may be another information. For example, in a case where the histogram is determined to show a bimodal distribution (for example, in a case where the histogram has a shape as illustrated in FIG. 5), comment creator 27 may create a comment such as “The CPAP device seems to be often used for XX hours and YY hours.” and transmit the comment to PC 30. Here, comment creator 27 may determine not only the bimodality but also the multimodality of the histogram, and may transmit peak occurrence times as a comment to PC 30.

In addition, for example, in a case where the histogram is determined to show a unimodal distribution (for example, in a case where the histogram has a shape as illustrated in FIG. 4), comment creator 27 may create a comment such as “The CPAP device seems to be often used for XX hours.” and transmit the comment to PC 30.

### 3 Summary

As described above, for example, the respiratory treatment support system according to the embodiment may take the following forms.

3-1 One form of the respiratory treatment support system according to the embodiment is respiratory treatment support system 100 including: a respiratory treatment device (CPAP device 10), server 20 communicably connected to the respiratory treatment device (CPAP device 10), and an information terminal (PC 30) communicably connected to server 20, in which server 20 receives operational information of the respiratory treatment device (CPAP device 10) from the respiratory treatment device (CPAP device 10), stores the operational information, calculates a statistical value and a histogram of the operational information, and can transmit the statistical value and the histogram of the operational information to the information terminal (PC 30).

Accordingly, it is possible to realize respiratory treatment support system 100 that enables a healthcare professional such as a physician to more accurately recognize the operation of the respiratory treatment device.

For example, in the conventional respiratory treatment support system, only statistical values are provided and displayed on the information terminal (PC 30), and thus when, for example, any one of the mean value or the median value is displayed as the statistical value, the physician has difficulty recognizing how much the provided statistical value can be trusted. As a result, it has been difficult to accurately recognize the operation of the respiratory treatment device. In contrast, according to respiratory treatment support system 100 according to the present embodiment, both the statistical value and the histogram can be displayed on the information terminal (PC 30), and thus the healthcare professional such as the physician can recognize, from the relationship between the statistical values and the histogram, which statistical value is an appropriate statistical value for determining the treatment plan.

In particular, in terms of the usage time of CPAP device 10, there is a unique situation in which bedtime and wake-up time vary greatly depending on the lifestyle of the patient, and the mean value and the median value as the statistical values tend to diverge . For example, in a case where the statistical value (mean value or median value) having a shorter usage time is displayed, the physician may erroneously recognize that the usage time is short even though the usage time is actually sufficient, and may give an erroneous instruction to the patient to increase the sleep time. According to the configuration of the present embodiment, the statistical values and the histogram can be displayed, and thus such an erroneous recognition and an erroneous instruction by the physician can be effectively avoided.

In addition, the healthcare professional such as the physician determines the necessity of the mask replacement based on the leak amount indicating how much air leaks from the mask of the patient. Specifically, in a case where the leak amount is high, the physician instructs the patient to change the mask. Erroneous determination and instruction cause unnecessary mask replacement. The healthcare professional can determine the necessity of the mask replacement based on the statistical values and the histogram for the leak amount as well, the erroneous determination of the necessity of the mask replacement can be suppressed. For example, when there is data with a relatively low leak amount in the leak amount histogram, it can be determined that the mask does not need to be changed. In addition, in a case where the mean value is less than a threshold value, it may be determined that the mask does not need to be changed.

3-2 One form of the respiratory treatment support system according to the embodiment is as follows. In <3-1>, the statistical value includes a mean value related to the operational information and a median value related to the operational information, and the information terminal (PC 30) is capable of selecting at least one of the mean value and/or the median value calculated by server 20, and receives, from server 20, the selected at least one of the mean value and/or median value to display the value.

3-3 One form of the respiratory treatment support system according to the embodiment is as follows. In <3-1>, the statistical value includes a plurality of percentile values related to the operational information, and the information terminal (PC 30) is capable of selecting at least one percentile value from among the plurality of percentile values, and receives the selected one percentile value from server 20 to display the value.

3-4 One form of the respiratory treatment support system according to the embodiment is as follows. In <3-1>, the statistical value includes a mean value related to the operational information, a median value related to the operational information, and a plurality of percentile values related to the operational information, and the information terminal (PC 30) displays the histogram together with the mean value and the median value and/or together with the plurality of percentile values on the same screen.

3-5 One form of the respiratory treatment support system according to the embodiment is as follows. In <3-4>, the information terminal (PC 30) is capable of selecting, on the screen, either the mean value or the median value, or at least one percentile value from the plurality of percentile values, and displays the selected value as a statistical value.

3-6 One form of the respiratory treatment support system according to the embodiment is as follows. In <3-1>, server 20 further transmits comment information based on a frequency distribution of the histogram to the information terminal (PC 30).

3-7 One form of the respiratory treatment support system according to the embodiment is as follows. In <3-6>, the comment information transmitted from server 20 to the information terminal (PC 30) is information that prompts a user of the information terminal (PC 30) to select and display the histogram.

3-8 One form of the respiratory treatment support system according to the embodiment is as follows. In <3-1> to <3-7>, the respiratory treatment device is a continuous positive airway pressure device (CPAP device 10), and the operational information is at least one piece of information among information on a usage duration, pressure, or leak of the continuous positive airway pressure device (CPAP device 10).

3-9 One form of the server according to the embodiment is server 20 used in respiratory treatment support system 100 including: a respiratory treatment device (CPAP device 10); server 20 communicably connected to the respiratory treatment device (CPAP device 10); and an information terminal (PC 30) communicably connected to server 20, in which server 20 receives operational information of the respiratory treatment device (CPAP device 10) from the respiratory treatment device (CPAP device 10), stores the operational information, calculates a statistical value and a histogram of the operational information, and can transmit the statistical value and the histogram of the operational information to the information terminal (PC 30).

3-10 One form of the display device according to the embodiment is a display device used in respiratory treatment support system 100 including: a respiratory treatment device (CPAP device 10); server 20 communicably connected to the respiratory treatment device (CPAP device 10); and an information terminal (PC 30) communicably connected to server 20, in which the display device receives a statistical value and a histogram of operational information of the respiratory treatment device (CPAP device 10) from server 20, and can display the statistical value and the histogram. The display device may be, for example, the information terminal (PC 30), or may be another device connected to server 20.

### 4 Other Embodiments

The above-described embodiment merely shows one example of specific embodiment of the present invention, and the technical scope of the present invention should not be construed as being limited thereto. That is, the present invention can be implemented in various forms without departing from the gist or the main features of the present invention.

The present disclosure includes a communication method used in a respiratory treatment support system including: a respiratory treatment device; a server communicably connected to the respiratory treatment device; and an information terminal communicably connected to the server. The communication method includes: receiving, by the server, operational information on the respiratory treatment device from the respiratory treatment device; storing, by the server, the operational information and calculating a statistical value and a histogram of the operational information; and transmitting, by the server, the statistical value and the histogram of the operational information to the information terminal.

The present disclosure includes a program used in a respiratory treatment support system including: a respiratory treatment device; a server communicably connected to the respiratory treatment device; and an information terminal communicably connected to the server. The program causes the server to execute a process of receiving operational information on the respiratory treatment device from the respiratory treatment device, a process of calculating a statistical value and a histogram of the operational information, and a process of transmitting the statistical value and the histogram of the operational information to the information terminal.

The present disclosure includes a storage medium used in a respiratory treatment support system including: a respiratory treatment device; a server communicably connected to the respiratory treatment device; and an information terminal communicably connected to the server. The storage medium stores a program causing the server to execute a process of receiving operational information on the respiratory treatment device from the respiratory treatment device, a process of calculating a statistical value and a histogram of the operational information, and a process of transmitting the statistical value and the histogram of the operational information to the information terminal.

In the above-described embodiment, the case where CPAP device 10 is used as the respiratory treatment device has been described, but the present invention is not limited thereto, and the present invention can also be applied to a respiratory treatment support system using another respiratory treatment device such as an oxygen concentrator or a ventilator.

In the above-described embodiment, the case where the information terminal is a personal computer (PC 30) has been described, but the present invention is not limited thereto, and the information terminal may be a tablet-type information terminal, a smartphone, or the like.

In the above-described embodiment, the 90th percentile and the 95th percentile have been described as the plurality of selectable percentiles, but the present invention is not limited thereto, and other percentiles may be used.

In the above-described embodiment, FIGS. 4 and 5 are illustrated as the display examples of the histogram, but the histogram may be, for example, displayed alone in a pop-up, or may be displayed together with the mean value or the median value as in FIGS. 4 and 5.

According to the respiratory treatment support system according to the present embodiment, when, for example, collaborating with other servers, numerical data acquired from a plurality of CPAP devices, servers, or the like from different manufacturers can be displayed on the terminal PC with unified units. Furthermore, according to the respiratory treatment support system according to the present embodiment, when, for example, outputting numerical data to another server, numerical data adapted to the server’s required specifications (units or the like) can be output.

### Reference Signs List

10 (10-1 to 10-n) CPAP device

20 Server

21 Storage

22 Mean value calculator

23 Median value calculator

24 90th percentile calculator

25 95th percentile calculator

26 Histogram creator

27 Comment creator

100 Respiratory treatment support system

What is claimed is:

1. A respiratory treatment support system, comprising:
a respiratory treatment device;
a server communicably connected to the respiratory treatment device; and
an information terminal communicably connected to the server, wherein
the server receives operational information on the respiratory treatment device from the respiratory treatment device,
the server stores the operational information and calculates a statistical value and a histogram of the operational information, and
the server is capable of transmitting the statistical value and the histogram to the information terminal.

2. The respiratory treatment support system according to claim 1, wherein
the statistical value includes a mean value related to the operational information and a median value related to the operational information, and
the information terminal is capable of selecting at least one of the mean value and/or the median value calculated by the server, and
the information terminal receives, from the server, at least one of the selected mean value and/or the selected median value, and displays at least one of the selected mean value and/or the selected median value.

3. The respiratory treatment support system according to claim 2, wherein
the statistical value includes a plurality of percentile values related to the operational information, and
the information terminal is capable of selecting at least one percentile value from among the plurality of percentile values, and
the information terminal receives the selected one percentile value from the server, and displays the selected one percentile value.

4. The respiratory treatment support system according to claim 1, wherein
the statistical value includes a mean value related to the operational information, a median value related to the operational information, and a plurality of percentile values related to the operational information, and
the information terminal displays the histogram together with the mean value and the median value and/or together with the plurality of percentile values on a same screen.

5. The respiratory treatment support system according to claim 4, wherein
the information terminal is capable of selecting, on the screen, either the mean value or the median value, or at least one percentile value from the plurality of percentile values, and displays the selected value as a statistical value.

6. The respiratory treatment support system according to claim 1, wherein
the server further transmits comment information based on a frequency distribution of the histogram to the information terminal.

7. The respiratory treatment support system according to claim 6, wherein
the comment information transmitted from the server to the information terminal is information that prompts a user of the information terminal to select and display the histogram.

8. The respiratory treatment support system according to claim 1, wherein
the respiratory treatment device is a continuous positive airway pressure device, and
the operational information is at least one piece of information among information on a usage duration, pressure, and leak of the continuous positive airway pressure device.

9. A server used in a respiratory treatment support system including: a respiratory treatment device; the server communicably connected to the respiratory treatment device; and an information terminal communicably connected to the server, wherein
the server receives operational information on the respiratory treatment device from the respiratory treatment device,
the server stores the operational information and calculate a statistical value and a histogram of the operational information, and
the server is capable of transmitting the statistical value and the histogram to the information terminal.

10. A display device used in a respiratory treatment support system including: a respiratory treatment device; a server communicably connected to the respiratory treatment device; and an information terminal communicably connected to the server, wherein
the display device receives a statistical value and a histogram of operational information on the respiratory treatment device from the server, and
the display device is capable of displaying the statistical value and the histogram.
